(19) United States
(12) Patent Application Publication
(10) Pub. No.: US 2005/0095666 A1
(43) Pub. Date: May 5, 2005

(54) Apparatus and methods for tissue preparation

(76) Inventors: Sameer Jhavar, London (GB); Stefan Reinsberg, London (GB); Andrew Jackson, London (GB); Craig Cummings, London (GB); Rosalind Eeles, London (GB); Alison Falconer, London (GB); Martin Leach, London (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/902,547

(22) Filed: Jul. 29, 2004

(30) Foreign Application Priority Data
Aug. 1, 2003 (GB) GB-A-0318125.2

(51) Int. Cl. A61B 18/18; G01N 33/48; B26D 5/08

(52) U.S. Cl. 435/40.52; 83/523

(57) ABSTRACT

The present invention features a multi-bladed cutter and a cradle adapted to grip a tissue sample 10 during cutting. The cutter has a series 4 of juxtaposed blades 5 and the cradle has a series of slits 13 adapted to receive the blades during cutting. The cradle may have opposing curved walls 8,9 which may be brought together to clasp the sample. The apparatus is suitable for cutting tissue such as prostate tissue, which is difficult to section. The apparatus is particularly suited to cutting tissue where chemical fixing is undesirable because nucleic acids are to be sampled from the tissue.

### FIELD OF THE INVENTION

The present invention relates to apparatus and methods for tissue preparation, in particular cutting tissue by slicing. The present invention is particularly useful where chemical fixing of the tissue is undesirable, for example where nucleic acids such as DNA or RNA are to be sampled from the tissue. The present invention may also be useful for preparing tissues that are difficult to section or slice without chemical fixing beforehand, such as prostate tissue.

### BACKGROUND OF THE INVENTION

Prostate cancer is the second most common cancer in men and accounts for over 9000 deaths per year in the United Kingdom. Treatment of prostate cancer has considerable morbidity, for example impotence and known side effects of radiotherapy. Diagnosis can be accomplished by testing of an appropriate tissue sample, for example using histology. Additionally, techniques such as Magnetic Resonance Imaging (MRI) or other medical imaging techniques may be used in diagnosis. MRI and laboratory techniques using for example microarray technology are sometimes used to investigate the disease further. Accurate diagnosis is essential to ensure that a patient does not suffer the consequences of unnecessary treatment.

Conventional histological preparation of a tissue sample involves 48-72 hours of formalin fixation, slicing the sample, embedding the slices in paraffin, sectioning and staining of the sections. The formalin fixation step of the conventional method is necessary to make the tissue firmer for cutting.

There is a particular need to fix certain types of tissue before cutting, for example a prostate gland. Such tissues have a semi-solid consistency and tend to deform or even disintegrate when cut, preventing uniform slicing of the tissue. Certain tissues may be difficult to cut owing to their structure. For example, when the capsule of a prostate gland is nicked, the pressure of the hypertrophic nodules within may cause the tissue to bulge through the nick, making further slicing extremely difficult without destroying the integrity of the prostate gland.

A disadvantage, however, of the conventional method is that the treated tissue is unsuitable for certain research applications or diagnostic techniques. In particular, the conventional treatment method destroys nucleic acids such as RNA in the tissue, so that the in-situ expression profile is lost. Furthermore, the amount of available DNA in the sample is significantly reduced compared to an untreated tissue sample. Once the tissue has been prepared for histology it is therefore unusable for techniques that measure quantitatively levels of particular molecules in the tissue.

Locating a piece of tissue that actually contains the suspected diseased tissue is also problematic. The tissue sample that is tested may not contain the diseased tissue (e.g. tumour). For example, if a prostate gland is removed and prepared for histology, the section tested may not, in contrast to the rest of the gland, contain tumour.

Furthermore, it is not possible to correlate the tissue sections to medical images, usually radiological images, which may have been obtained earlier in the diagnosis process, to assist and/or confirm the diagnosis. The radiological images may comprise virtual slices of tissue that potentially could be matched to a corresponding slice of tissue sample. However, it is extremely difficult and time consuming to produce tissue slices that match the virtual slices.

U.S. Pat. No. 4,852,256, GB506594 and U.S. Pat. No. 4,937,938 show cutters for slicing solid foodstuffs. In U.S. Pat. No. 4,852,256 and GB506594, apparatus is provided that has a base for a foodstuff (e.g. a hardboiled egg). The base is hinged to an opposing multi-bladed cutter, which has blades that are received in slits in the base during cutting of the foodstuff. The base provides a platform on which the foodstuff rests during cutting. In U.S. Pat. No. 4,937,938 an egg is placed on the blades of a multi-bladed cutter and a presser, hinged to the cutter, forces the egg against the blades.

U.S. Pat. No. 3,816,919 relates to an elongate channel-shaped member for carrying a nerve. The apparatus is useful for progressively cutting back a diseased nerve using a single blade to reveal healthy nerve tissue. A peripheral region of the channel has a slit that allows the end of the nerve to be wrapped in silicone rubber during cutting with the blade.

U.S. Pat. No. 5,662,661 and U.S. Pat. No. 5,989,273 relate to multi-bladed cutters for removing scalp sections. No tissue support means is provided as the apparatus is designed to remove strips of tissue directly from a patient.

### SUMMARY OF THE INVENTION

Broadly, the present invention provides a tissue slicing apparatus comprising a tissue cutting means comprising a series of juxtaposed blades linked to a support. The apparatus may further comprise a cradle adapted to grip a tissue sample while it is cut with the tissue cutting means. The cradle can help to prevent the tissue sample deforming during sample preparation, even using an untreated or fragile sample, such as a prostate. As the tissue slices are produced together and in a defined orientation, the apparatus can also help to correlate tissue sections with diagnostic images.

Accordingly, in a first aspect, the present invention provides a tissue slicing apparatus comprising:
tissue cutting means comprising a series of juxtaposed blades linked to a support; and
a cradle adapted to grip a tissue sample, the cradle having a series of slits adapted to receive the blades of the tissue cutting means, such that, in use, a tissue sample is gripped in the cradle and the blades are movable in the slits to cut the tissue sample into slices.

The tissue can therefore be supported during cutting, removing the need to fix the tissue beforehand. Preferably, the blades are planar. Preferably the blades are metal, for example stainless steel.

The series of blades may contain two or more blades, for example five, six, seven, eight, nine or ten blades. The series may, for example, comprise or consist essentially of at least five blades. Preferably, the series comprises or consists essentially of seven blades. The series of blades therefore allows the sample to be cut simultaneously into multiple slices, reducing or eliminating the need to fix the tissue beforehand. The time required to cut the sample into multiple slices is reduced.

Preferably, the blades in the series of blades are substantially parallel. Preferably, the blades are spaced substantially evenly apart. The sample can therefore be cut into multiple, uniform slices, even if the tissue sample in the cradle is of a semi-solid nature. Spaces between the blades may be at least 3 mm but any arrangement is suitable where the blades are spaced to produce tissue slices for examination. For example, the spacing between the blades may be 4 mm or 5 mm or more.

The length of each blade is preferably suitable for slicing completely through a prostate gland, most preferably a human prostate gland. Ideally, the number and spacing of the blades is such that a whole prostate gland can be cut simultaneously into slices with the cutting means. The depth of each blade may be at least about 10 mm.

The blades in the series may conveniently be uniform. For example, the blades may be of approximately the same length and/or depth and/or shape. The cutting edges of the blades preferably lie in the same plane.

The tissue cutting means may include a handle. The blades may extend from the handle, preferably substantially axially. The handle may be metal, for example stainless steel. The axis of the handle and the axis of the cutting edge of a blade in the series may be offset by at least the depth of the blade such that the cutting edge of the blade is further from the axis of the handle than the opposing edge of the blade. If, during cutting of the tissue, the blades slice through the tissue and abut the underlying support, the offset ensures that the handle is clear of the underlying support.

Preferably the number of blades in the series is adjustable. The spacing between the blades may be adjustable. Advantageously, an adjustable cutting means allows different types and sizes of tissue to be cut.

The blades may be detachable from the support. Preferably, the whole series of blades is detachable from the support as a complete unit. If the blades are detachable as a single unit, an alternate series of blades comprising, for example, additional blades, may be attached. There may be several modular units available, each unit having a different arrangement of blades, and attachable to the support. Additionally or alternatively, each blade may be detachable from the support separately. Advantageously, detachable blades allow easier cleaning or sterilisation of the apparatus. Blades can also be replaced easily if they become damaged or blunt.

Conveniently, the cradle may include a well or chamber for a tissue sample. Preferably, the cradle includes means to allow the user to exert a gripping force on a sample in the well or chamber by decreasing a dimension of the well or chamber.

Preferably, the slits are in opposing walls of the well or chamber. Preferably, the distance between said walls is said dimension.

The cradle may include a handle that is movable to cause said dimension to decrease. The cradle may include a first handle and a second handle, one of which is movable relative to the other handle to cause said dimension to decrease.

The cradle may comprise a base and a series of rods, the base having a plurality of formations, each of which is adapted to cooperate with a rod such that the rods are attachable to the base at an oblique angle relative to the base to form a chamber for the tissue sample.

The space between adjacent rods may act as a slit and receive a blade of the tissue cutting means. Preferably, there are opposing rows of rods. A blade of the tissue cutting means may pass between the space between adjacent rods in opposing rows. Advantageously, the rods may be spaced so as to provide slits that receive the blades of the cutting means snugly, with little or no movement of the blade laterally relative to the direction of cutting.

Preferably the cradle further comprises at least one side wall that extends between an end rod of the opposing rows of rods to form the tissue chamber. Preferably the cradle comprises two side walls that extend between the end rod of the opposing rows of rods to form the tissue chamber.

The provision of a side wall may ensure that at least one end section of the tissue to be sliced (those sections that lie outside of the first and last blade in the series of blades of the cutting means) are of equivalent thickness to the other slices (e.g. 4 mm). The first rod in each row is spaced a fixed distance from the side wall, which distance is ideally equivalent to the distance between rods. The end section of the tissue will therefore be of equivalent thickness to sections cut from the central portion of the tissue.

The base may be provided with a series of holes, each hole being dimensioned to receive a rod. Alternatively, the base may comprise a series of projections adapted to be received in a complementary hole in the base of a rod.

The base may be provided with a channel into which a side wall may be slidably received. Preferably, the base is provided with two channels spaced so as to receive opposing side walls. The side walls may be attached to the base using the same complementary formations as the rods.

Ideally, the rods are detachably attachable to the base. The chamber may therefore be varied in dimension and/or shape according to the particular being sliced. Preferably, the at least one side wall is detachably attachable to the base. The cradle may be dismantled to facilitate cleaning and or sterilisation of the cradle.

The row of rods may be a straight line or may be curved to account for the contours of the tissue sample to be sliced. There are preferably more formations on the base than there are rods to allow the size and shape of the chamber to be varied by altering the position of the rods on the base. The rods are preferably arranged so as to grip and thereby support the tissue sample, preferably without deforming the tissue sample.

The rods are preferably substantially cylindrical but may be any suitable cross section. There may be at least ten rods, preferably arranged in opposing rows. There may be two opposing rows having at least nine rods per row.

Preferably, at least some of the rods include contours on the surface of the rod, such as grooves, channels, protrusions or striations. The contours are adapted to increase friction between the rod and the tissue sample, and thereby reduce or prevent rotation of the tissue sample in the chamber during cutting. The tissue sample is preferably unfixed.

In a further aspect, the present invention provides a kit comprising tissue slicing apparatus according to the first aspect of the invention, and at least one further series of blades, the further series being attachable to the support in place of said series of blades.

Preferably, the further series of blades differs from said series of blades in the number and/or dimension of blades. The kit may therefore allow different types and sizes of tissue sample to be sliced.

The further series of blades may have a different spacing of blades from said series of blades. The kit may further comprise a second cradle having slits adapted to receive the blades of said further series of blades. The kit may include the cradle described above, which uses rods to adjust the size and shape of the tissue chamber and to vary the distance between slits. The kit may therefore allow slices of varying thickness to be cut.

In a further aspect, the present invention provides a method of slicing a tissue sample using a tissue slicing apparatus as described herein, which method includes:
(a) gripping said tissue sample in said cradle;
(b) using said cutting means to cut simultaneously said tissue sample into slices; and
(c) removing said slices from said cradle.

The tissue is preferably a prostate gland, or a portion thereof, although the apparatus and methods are suitable for other types of tissue. The gland may have been removed from a patient in a prostatectomy. As the tissue is cut simultaneously into slices and is supported during cutting, fixing of the tissue is not necessary. Analysis on the slices using, for example, techniques that examine molecular contents of the slice may thereafter be performed on the slices e.g. microarray techniques using RNA extracted from the slice. The analysis may comprise techniques for detecting or investigating cancer in the slice.

As the slices may be produced in a single cutting action, the time from extracting the tissue sample to storing the tissue slices is reduced compared to methods where slices are cut progressively from the tissue sample. The slices may therefore be stored rapidly to preserve the integrity and/or molecular content of the tissue. Many diagnosis techniques require tissue samples that have been appropriately stored soon after the sample was removed from the patient. Proper storage is essential in order to preserve tissue integrity for histology techniques (e.g. histopathology), and molecular content such as DNA and/or RNA.

Advantageously, fixing of individual slices produced by the method will be considerably quicker than the prior art method where the whole gland is fixed. Each slice can be stored flat in a storage cassette, which prevents warping of the shape of the slice. A slice that maintains a substantially flat shape is easier to section and is usually closer in shape to virtual slices of the tissue.

In a further aspect, the present invention provides a method of preparing tissue slices from an unfixed tissue sample, said method including:
a) following steps (a)-(c) of the above method;
b) fixing only certain slices of the sample for microscopy; and
c) storing the slices that are not fixed in conditions suitable to preserve said sections.

The nucleic acid content (e.g. RNA) of selected samples (for example tumour and normal tissue) from slices that are not fixed may be determined and correlated with histological data from stained sections prepared from adjacent fixed tissue slices. The said conditions may be a temperature low enough to preserve the slice. Alternate slices in the sample may be fixed, the adjacent sections being stored under suitable preserving conditions. The tissue is preferably a prostate gland, or a portion thereof.

In a further aspect, the present invention provides a method of diagnosing a medical condition comprising:
following steps a) to c) of the method of preparing tissue slices, above;
microscopy performed on the fixed slices to determine slice(s) afflicted by the said condition; and/or
retrieving slice(s) adjacent the afflicted slice(s); and taking, from a said adjacent slice, a sample of tissue; and
performing further analysis on the sample.

The medical condition may be cancer. The results of the further analysis, for example microarray analysis, may be correlated with the microscopy results of the adjacent slice. Comparison of data from different techniques performed on neighbouring slices may provide a more accurate diagnosis, or allow improvement of the predictive power of a technique.

In a further aspect, the present invention provides a method of producing tissue slices that correspond to virtual slices from medical imaging of the tissue, the method comprising:
a) obtaining a sample of said tissue;
b) cutting the tissue using the apparatus according to the first or second aspect of the invention to produce said tissue slices, such that the slice width matches the width of the virtual slices and the orientation of slices through the tissue matches the orientation of the virtual slices.

Medical imaging is preferably radiological imaging, such as MRI. The medical imaging may be ultrasound imaging.

Advantageously, a tissue slice produced by this method can be matched with the corresponding virtual slice. The number of tissue slices does not necessary have to be the same as the number of virtual slices; the medical imaging data may highlight a subsection of, for example prostate gland, that merits further study, so that only a portion of the gland needs to be cut into slices. However, if the slice width and orientation of the slices through the tissue sample match the width and orientation of the virtual slices, comparisons between virtual and real slices can be made.

The method further provides, as a further aspect, a method that facilitates locating a region of interest in a sample of tissue, which region has been identified from medical imaging of said tissue, the method comprising:
a) performing steps a) and b) of the method of producing tissue slices that correspond to virtual slices, defined above;
b) obtaining an image of a said tissue slice;
c) establishing a mapping function between the image and the corresponding virtual slice of the medical imaging data; and
d) using said mapping function to locate said region in the tissue slice.

The image is preferably a photograph. Preferably the photograph is taken using a digital camera to produce a digital image of said tissue slice. Preferably, the digital image is of sufficient resolution to allow a pixel-to-pixel mapping between the image and the corresponding virtual slice from the medical imaging data.

Preferably, the image and the corresponding virtual slice are aligned and/or overlaid, and tissue landmarks are used to establish the mapping. The tissue is preferably a prostate gland or a portion thereof. The landmarks may include any one or more of: capsule, urethra, ejaculatory duct, and seminal vesicle(s).

The mapping function allows a location in one of the real or virtual slice to be translated easily to the other of the equivalent real or virtual slice. Predictions based on the medical imaging data can therefore be correlated and/or compared with empirical studies on the corresponding tissue slice. Comparing data may allow a prediction based on medical imaging data to be confirmed.

In a further aspect, the present invention provides a method of diagnosing disease in a patient comprising:
obtaining medical imaging data of a tissue of interest;
performing steps (a)-(c) of the method defined above;
obtaining histological data from the tissue slice; and
using the mapping to compare and/or correlate the histological data and the medical imaging data to assess the health of the tissue.

The medical imaging data is preferably radiological data, most preferably magnetic resonance image (MRI) data. The MRI data may comprise any one or more of: dynamic magnetic resonance data; high-resolution morphology data; diffusion weighted images; and secondary data.

Further research may be performed on the slice, such as microarray techniques.

In a still further aspect of the invention, there is provided a cradle adapted to grip a tissue sample during tissue sectioning, which cradle comprises a base and a series of rods, the base having a plurality of formations, each of which is adapted to cooperate with a rod such that the rods are attachable to the base at an oblique angle relative to the base to form a tissue sample chamber, and wherein the dimensions of the tissue sample chamber are adjustable to match contours of the tissue sample by varying the position of attachment of the rods on the base.

The cradle may include features as set out above. For example, at least some of the rods may include contours on the surface of the rod to increase friction between the rod and the tissue sample. The tissue sample is preferably unfixed.

Embodiments of the invention will now be described, by way of example only, with reference to the accompanying drawings.

### BRIEF DESCRIPTION OF THE FIGURES

FIG. 1 shows a cutting means of the present invention;

FIG. 2 shows a cradle of the present invention gripping a prostate gland;

FIG. 3 shows cutting of a whole prostate gland using apparatus according to the present invention;

FIG. 4 shows a further cradle of the present invention; and

FIG. 5 shows a further cradle of the present invention having just one side wall.

### DETAILED DESCRIPTION

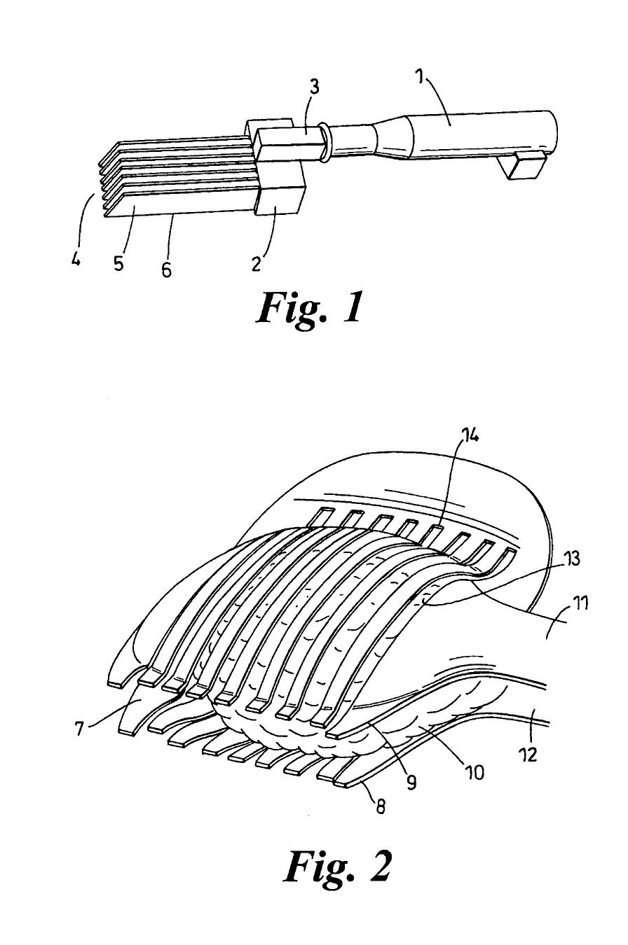

Cutting means of the present invention is shown in FIG. 1. The whole apparatus is of metal. The apparatus has an elongate handle 1, which thickens at one end to provide a handgrip. A blade support includes a support block 2 and a cuboid piece 3 attached at the other end of the handle. A series of blades, indicated generally at 4, extend from the support block in approximately the same direction as the axis of the handle.

The series of blades consists of seven planar blades, each planar blade 5 having a cutting edge 6. The blades are identical and their cutting edges lie in the same plane. The blades are individually detachable from the support block and the blade support is detachable from the handle.

The cutting edges 6 of the blades 5 are offset from the axis of the handle. When cutting edges abut an underlying surface, the offset provides, between the underside of the handle and the surface, some clearance for the user's fingers.

A cradle adapted to support and grip a tissue sample, as shown in FIG. 2, has a tissue gripping chamber, indicated generally at 7, defined by a pair of opposing walls 8, 9. Each wall 8, 9 is curved to define a recess (not shown).

When the walls are brought together, opposing recesses form the chamber 7. A prostate gland 10 is shown in the chamber 7, being gripped by the walls 8,9.

A handle 11 or 12 extends from each wall. The handles are hinged (not shown) at the ends distal to the wall. The cradle resembles tongs adapted to clasp the prostate gland 10, with walls 8,9 forming a clasping portion. Bringing together the walls effects a gripping force on the prostate gland in the chamber. The extent of the gripping force depends on the force used to bring together the handles and is therefore variable by the user.

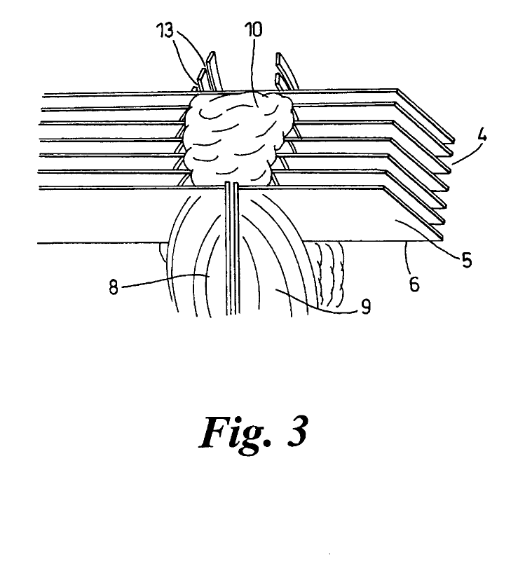

Each wall has slits 13. Each slit in a wall is aligned with a slit in the opposing wall such the aligned slits can receive a blade from the cutting means as the blade passes through the chamber. The whole prostate gland can therefore be placed in the chamber, gripped by the walls 8,9 and cut using the cutting means by sliding each blade 5, cutting edge 6 first, in its corresponding slit 13 until the cutting edge 6 abuts the slit end 14. The cutting process is shown in FIG. 3. The slits extend beyond the curved portion of the walls, i.e. beyond the chamber, to ensure that each cutting edge 6 passes completely through the prostate gland 10 in the chamber before each blade abuts its slit end 14.

A whole prostate gland can therefore be cut into slices in a single cutting action. The morphology and semi-solid consistency of the prostate gland would normally cause the gland to deform or disintegrate if cut, but the cradle grips and supports the gland as the blades slide through the gland during cutting. The cutting process is considerably quicker and easier than slicing fixed tissue using a single blade.

The complete labelling and slicing procedure can be completed within about 15-20 minutes. Alternate slices are stored at low temperature and/or in suitable media for preserving RNA and DNA in the sample. An advantage of such rapid slicing is capturing the in situ gene expression profile of tumour cells in the sample.

Once the gland has been cut into slices, samples may be taken from the apex and base of the gland. The slices are typically 3-4 mm thick. Alternate slices are stored in fenestrated plastic containers/cassettes that are suitable for formalin fixation. As formalin must infuse through the whole tissue, fixing individual slices is considerably quicker than fixing the whole gland (usually about 40-50 mm thick). Furthermore, the slices can remain flat in the cassette using foam gel pads, which prevents warping of the slice. The whole process, from sampling to having prepared slices, takes about 1-2 days, instead of 3-4 days using conventional methods. The untreated slices have well maintained histopathology if they are sampled immediately after cutting or stored in suitable conditions (and sampled later).

The treated slices undergo histopathology tests to detect regions of tumour growth. The untreated slice adjacent the fixed slice will be similar, perhaps identical, to the histopathology slice, and so the tumour is bored out of the untreated slice and samples taken for further research. Optionally, histopathological studies can be performed on more than one treated slice to confirm the presence and location of a tumour in the slices. Tumour cells in the untreated slice sandwiched between the two histopathology slices can therefore be easily located.

Once slicing is finished, the blades can be detached from the cutting means and the apparatus cleaned and sterilised as necessary.

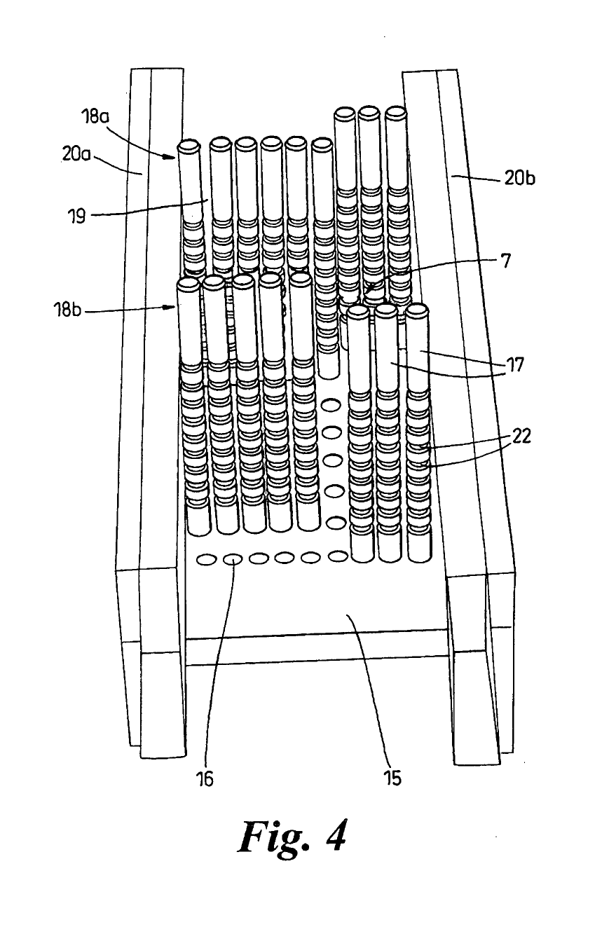

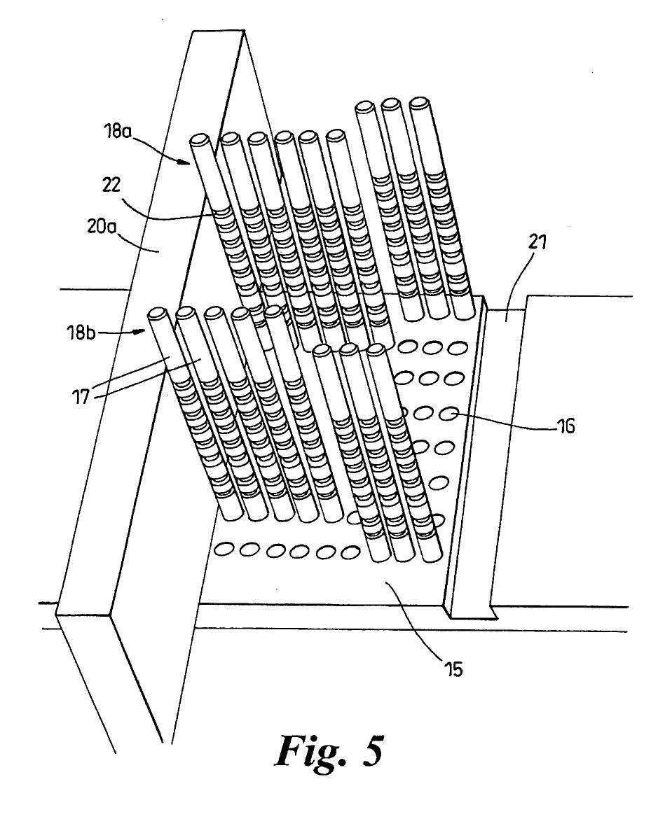

The cradles of FIGS. 4 and 5 are alternate or further cradles for use with the cutting means of the invention. The cradle comprises a base 15 having rows of holes 16 therein. In each row the holes are 4 mm apart. Brass rods 17 are snugly located within the holes to form two opposing rows of rods, 18a and 18b. The spaces 19 between adjacent rods in opposing rows act as slits for the blades 5 of the cutting means. The slits are therefore spaced 4 mm apart by the rods.

The cradle also comprises a pair of opposing side walls, 20a and 20b, in the form of end plates, which run between opposing end rods to create a chamber, indicated generally at 7, for the tissue sample. The base 15 and the end plates, 20a and 20b are of Perspex.

FIG. 5 shows a cradle with the right-hand end plate detached from the base. A slot or channel 21 is provided in the base to allow slidable attachment and detachment of the end plate.

In use, an unfixed, ideally freshly removed, prostate gland is placed on its posterior surface on the base and either the apical or basal pole of the prostate gland is in contact with an end plate. If two end plates are used, each of the basal and apical poles of the prostate gland is in contact with and end plate.

The brass rods are placed in the holes to form two opposing rows of rods, 18a and 18b, which grip the prostate gland without deforming the prostate gland. The rods contact the prostate gland around the equatorial region of the gland.

Once secured in the cradle, the prostate gland can be cut as described above using the cutter means. The juxtaposed blades 5 of the cutter means are received in the slits formed between adjacent rods in opposing rows 18a and 18b. The end plates ensure that the end slices of tissue are also 4 mm in thickness. In the examples, slicing occurs perpendicular to the posterior surface of the prostate gland.

The rods are provided with circumferential grooves 22 to increase friction between the prostate gland and each rod. The increase in friction prevents rotation of the prostate gland in the cradle during cutting.

The cradle is easily assembled and disassembled for cleaning and sterilisation.

Use of the Invention with MRI Data

If a patient has been assessed using magnetic resonance image (MRI) data and considered at risk, a prostatectomy is performed and the prostate tested. The MRI data contains information about the function of the gland, and comprises a series of virtual slices through the gland. The gland is cut using the apparatus in FIG. 1 and FIG. 2 to produce slices that essentially match the MRI virtual slices. The matching slices provide an added dimension to diagnosis as clinical findings can be correlated with both MRI information (such as gland function) and any predictions about the prostate tumour drawn from the MRI data.

The gland is gripped in the cradle at such an orientation that the slits traverse the gland in approximately the same direction as the virtual slices through the gland. The gripping and supporting features of the cradle allow the sample to be held in place and cut to produce slices through the gland that are oriented the same as the virtual slices. The simultaneous production of parallel slices by the series of blades provides slices that closely match the virtual slices. The procedure is considerably quicker and more accurate than a series of slicing operations using a blade. If the slice width of the MRI apparatus is set to match the slice width of the cutting means (or if a series of blades is available that matches the virtual slice width) a set of physical slices are produced that match the virtual slices.

The slices are imaged using a high resolution digital camera on a macro stand. Slices or samples therefrom are fixed and stained and undergo histopathological testing. The high-resolution images can the be compared to the virtual slices, equivalent slices paired together and a mapping function developed to allow a location in one slice of the pair to be translated to the corresponding location in the other slice. During mapping, the image and the virtual slice are aligned or overlaid, and landmarks such as the urethra, seminal vesicles, the ejaculatory duct and the capsule compared to establish a pixel to pixel mapping between slices in a pair. The process of comparison is known as image registration.

Slices that are shown to have tumour growth, as identified by histopathology on the samples, are compared to the virtual slices. The region of tumour growth is marked on the image as the sample tested was excised from a known area of the slice. The tumourigenic region is translated to the virtual slice. This procedure may be performed on numerous slices so that a three dimensional picture of the tumour in the gland is built up, and compared to regions of interest and/or functional data shown in the original MRI data.

If a region of interest is identified in the gland from the MRI data, the suitable tissue slice can be identified, and, using the mapping function, a sample excised from the correct region. The MRI prediction or diagnosis can be verified or investigated using reliable histopathological and/or microarray techniques.

1. A tissue slicing apparatus comprising:
a tissue cutter comprising a series of juxtaposed blades linked to a support; and
a cradle adapted to grip a tissue sample, the cradle having a series of slits adapted to receive the blades of the tissue cutter, such that, in use, a tissue sample is gripped in the cradle and the blades are movable in the slits to cut the tissue sample into slices.

2. The tissue slicing apparatus according to claim 1, wherein the series of blades comprises at least five blades.

3. The tissue slicing apparatus according to claim 1, wherein the blades in the series of blades are substantially parallel and spaced substantially evenly apart.

4. The tissue slicing apparatus according to claim 1, wherein the length of each blade is suitable for slicing completely through a prostate gland.

5. The tissue slicing apparatus according to claim 1, wherein the number and spacing of the blades is such that a whole prostate gland can be cut simultaneously into slices with said cutter.

6. The tissue slicing apparatus according to claim 4, wherein the prostate gland is of human origin.

7. The tissue slicing apparatus according to claim 1, wherein the cradle includes a well or chamber for a tissue sample, the cradle being adapted to allow the user to exert a gripping force on a sample in the well or chamber by decreasing a dimension of the well or chamber.

8. The tissue slicing apparatus according to claim 7, wherein the slits are in opposing walls of the well or chamber, the distance between said walls being said dimension.

9. The tissue slicing apparatus according to claim 7, wherein the cradle comprises a base and a series of rods, the base having a plurality of formations, each of which is adapted to cooperate with a rod such that the rods are attachable to the base at an oblique angle relative to the base to form a chamber for the tissue sample.

10. The tissue slicing apparatus according to claim 9, wherein the rods are arranged as opposing rows of rods, and the cradle further comprises at least one side wall that extends between an end rod of each opposing row to form a tissue chamber.

11. The tissue slicing apparatus according to claim 9, wherein at least some of the rods include contours on the surface of the rod to increase friction between the rod and the tissue sample.

12. The tissue slicing apparatus according to claim 1, wherein the tissue cutter includes a handle and the blades extend from the handle.

13. The tissue slicing apparatus according to claim 12, wherein the axis of the handle and the axis of the cutting edge of a blade are offset to by at least the depth of the blade such that the cutting edge of the blade is further from the axis of the handle than the opposing edge of the blade.

14. The tissue slicing apparatus according to claim 1, wherein the series of blades is adjustable by altering one or more of: the number of blades, the spacing of the blades.

15. The tissue slicing apparatus according to claim 1, wherein the blades are detachable from the support.

16. The tissue slicing apparatus according to claim 15, wherein the series is detachable from the support as a complete unit.

17. A kit comprising tissue slicing apparatus according to claim 15, and at least one further series of blades, the further series being attachable to the support in place of said series of blades.

18. The kit according to claim 17, wherein said further series of blades differs from said series of blades in one or more of: the number of blades, the dimension of the blades.

19. The kit according to claim 17, wherein said further series of blades has a different spacing of blades from said series of blades and said kit comprises one or more of: a cradle adjustable to receive the blades of said further series of blades, a second cradle having slits adapted to receive the blades of said further series of blades.

20. A method of slicing a tissue sample using a tissue slicing apparatus according to claim 1, which method includes:
(a) gripping said tissue sample in said cradle;
(b) using said cutter to cut simultaneously said tissue sample into slices; and
(c) removing said slices from said cradle.

21. A method of preparing tissue slices from an unfixed tissue sample, said method comprising:
(a) following steps (a)-(c) of the method according to claim 20;
(b) fixing only certain slices of the sample for microscopy; and
(c) storing the slices that are not fixed in conditions suitable to preserve said slices.

22. The method according to claim 21, wherein alternate slices in the sliced sample are fixed.

23. A method of diagnosing a medical condition comprising:
(a) following steps a) to c) of claim 21;
(b) performing microscopy on the fixed slices to determine slice(s) afflicted by the said condition; and/or
(c) retrieving unfixed slice(s) adjacent the afflicted slice(s), and taking, from a said unfixed slice, a sample of tissue; and
(d) performing further diagnostic analysis on the sample taken in step (c), above.

24. A method of producing tissue slices that correspond to virtual slices from medical imaging of the tissue, the method comprising:
(a) obtaining a sample of said tissue; and
(b) cutting the tissue using the apparatus according to claim 1 to produce said tissue slices, wherein the slice width matches the width of the virtual slices and the orientation of slices through the tissue matches the orientation of the virtual slices.

25. A method that facilitates locating a region of interest in a sample of tissue, which region has been identified from medical image data of said tissue, the method comprising:
(a) performing steps (a) and (b) of claim 24;
(b) obtaining an image of a said tissue slice;
(c) establishing a mapping function between the said image and the corresponding virtual slice of the medical image data; and
(d) using said mapping function to locate the said region in the tissue slice.

26. The method according to claim 25, wherein said image is produced using a digital camera to produce a digital image of said tissue slice.

27. The method according to claim 26, wherein the digital image is of sufficient resolution to allow a pixel-to-pixel mapping between the image and the corresponding virtual slice from the medical image data.

28. The method according to claim 25, wherein the photograph and the corresponding virtual slice are aligned, and tissue landmarks are used to establish the mapping function.

29. The method according to claim 24, wherein the tissue is a prostate gland or a portion thereof.

30. The method according to claim 29, wherein the tissue is a prostate gland or a portion thereof, and the landmarks include any one or more of: capsule, urethra, ejaculatory duct, and seminal vesicle(s).

31. A method of diagnosing disease in a patient comprising:
(a) obtaining medical image data of a tissue of interest;
(b) performing steps a)-c) of claim 25;
(c) obtaining histological data from the tissue slice; and
(d) using the mapping function to compare and/or correlate the histological data and the medical image data to assess the health of the tissue.

32. The method according to claim 24, wherein the medical image data includes, or consists essentially of, magnetic resonance image (MRI) data.

33. The method according to claim 32, wherein the MRI data comprises any one or more of: dynamic magnetic resonance data; high-resolution morphology data; diffusion weighted images; and secondary data.

34. A cradle adapted to grip a tissue sample during tissue sectioning, which cradle comprises a base and a series of rods, the base having a plurality of formations, each of which is adapted to cooperate with a rod such that the rods are attachable to the base at an oblique angle relative to the base to form a tissue sample chamber, and wherein the dimensions of the tissue sample chamber are adjustable to match contours of the tissue sample by varying the position of attachment of the rods on the base.

35. A cradle according to claim 34, wherein at least some of the rods include contours on the surface of the rod to increase friction between the rod and the tissue sample.
